(19) United States
(12) Patent Application Publication
(10) Pub. No.: US 2003/0133835 A1
(43) Pub. Date: Jul. 17, 2003

(54) Intravenous oxygenator having an impeller

(76) Inventor: Brack G. Hattler, Pittsburgh, PA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/052,103

(22) Filed: Jan. 16, 2002

(51) Int. Cl. A61M 1/14; A61M 37/00

(52) U.S. Cl. 422/45; 604/6.14

(57) ABSTRACT

An intravenous oxygenator having hollow, gas-permeable fibers extending between a distal manifold and a proximal manifold that permit diffusion of gases between the blood vessel and interior of the fibers A rotatable support member extends through the proximal manifold and into the distal manifold. The support member has a lumen in communication with the distal manifold so that oxygen-containing gases flow through the support member, distal manifold, fibers, and proximal manifold. An impeller is attached to and rotated by the support member to enhance blood flow around the fibers.

### BACKGROUND OF THE INVENTION

1. Field of the Invention

The present invention relates generally to the field of intravenous oxygenators used to increase the oxygen level in a patient's blood. More specifically, the present invention discloses an intravenous oxygenator having an impeller to accelerate the exchange of gases between the oxygenator and the surrounding blood.

2. Statement of the Problem

Many types of blood oxygenators are well known in the art. For example, during open heart surgery, the patient is interconnected with an external oxygenator, commonly known as a heart-lung machine, which introduces oxygen into the blood system. Most types of oxygenators use a gas-permeable membrane. Blood flows along one side of the membrane and oxygen is supplied to the other side of the membrane. Given a sufficient pressure gradient between the oxygen supply and the blood, oxygen will diffuse through the membrane and into the blood. In addition, carbon dioxide in the blood will tend to diffuse from the blood into the interior of the membrane.

In other situations, a smaller implantable oxygenator may be sufficient to adequately supplement the patient's cardiopulmonary function by marginally increasing the oxygen content of the patient's blood. For example, patients suffering from emphysema, pneumonia, congestive heart failure, or other chronic lung disease often have blood oxygen partial pressures of approximately 40 torr. A relatively small increase of 10% to 20% is generally sufficient to adequately maintain the patient. An implantable oxygenator is a particularly desirable alternative in that it avoids the need to intubate the patient in such cases. In addition, temporary use of an implantable oxygenator is sufficient in many cases to tide the patient over an acute respiratory insult. Placing such patients on a conventional respirator is often the beginning of a progressive downhill spiral by damaging the patient's pulmonary tree and thereby causing greater dependence on the respirator.

Implantable oxygenators typically include a plurality of hollow gas-permeable membrane fibers which form a loop so that oxygen can be fed into one end of each fiber and carbon dioxide removed from the other end as a result of the cross-diffusion that takes place. The effective rate of diffusion in implantable oxygenators can be limited in some instances by the problem of “streaming” or “channeling”, wherein the blood stream establishes relatively stable patterns of flow around and through the oxygenator. Only portions of the fibers are exposed to a relatively high velocity, turbulent flow of blood. These conditions tend to increase cross-diffusion of oxygen and carbon dioxide. However, other portions of the fibers are exposed to a low velocity, laminar flow of blood which reduces diffusion of gases. Those portions of the fibers immediately adjacent to the regions of high blood flow may continue to experience high rates of diffusion, but the remaining portions of the fibers tend to have relatively low diffusion rates. Thus, the overall diffusion rate of the oxygenator can be substantially diminished by streaming.

The applicant's U.S. Pat. No. 5,271,743 discloses a percutaneous oxygenator having a plurality of hollow gas-permeable but liquid-impermeable fibers that form loops and are insertable into a blood vessel. Oxygen gas is fed into one end of the fibers and carbon dioxide is withdrawn from the opposite end of the fibers as a result of cross-diffusion of oxygen and carbon dioxide through the fiber walls when the oxygenator is positioned within the blood vessel. In one embodiment of the oxygenator, a system for agitating the blood is positioned within the loops formed by the fibers so that the linear flow of blood is disrupted and the blood is directed radially by the agitator to randomly move the fibers and thereby prevent streaming. For example, the agitator can be a rotating curved blade designed to disrupt the linear blood flow and redirect the flow into swirling radially-oriented patterns.

3. Solution to the Problem

None of the prior art references known to the applicant teach or suggest the specific structure of the present invention. In particular, the present oxygenator employs a rotating central shaft both to supply oxygen to a manifold at the distal ends of the fibers and to drive an impeller to agitate the blood adjacent to the fibers and thereby minimize streaming and maximize cross-diffusion of gases between the patient's blood stream and the oxygenator.

### SUMMARY OF THE INVENTION

This invention provides an intravenous oxygenator having a plurality of hollow gas-permeable fibers extending between a proximal manifold and a distal manifold. A hollow rotatable central shaft passes through the proximal manifold and extends into the distal manifold. Oxygen is supplied to the distal manifold via the central shaft, while a vacuum pump is connected to the proximal manifold. This induces a flow of oxygen through the gas-permeable fibers. Alternatively, oxygen can be supplied through the proximal manifold and suction can be applied via the central shaft and distal manifold. The oxygenator is inserted into a blood vessel so that when oxygen is drawn through the fibers, it will diffuse through the walls of the fibers and into the adjacent blood stream, while excess carbon dioxide in the blood will pass in a reverse or cross-diffusion pattern through the walls of the fibers into the interior thereof for removal from the fibers. An impeller or rotatable blade attached to the central shaft agitates the blood so as to disrupt the linear flow of blood and direct it radially in a convective swirling fashion to keep the fibers moving and optimally disposed for maximum gas diffusion.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a side cross-sectional view of the intravenous oxygenator.

FIG. 2 is a cross-sectional view of the intravenous oxygenator taken along lines 2-2 in FIG. 1.

FIG. 3 is a cross-sectional view of the intravenous oxygenator taken along lines 3-3 in FIG. 1

FIG. 4 is a side cross-sectional view of another embodiment of the intravenous oxygenator using a hollow flexible cable 141 as the central support member.

### DETAILED DESCRIPTION OF THE INVENTION

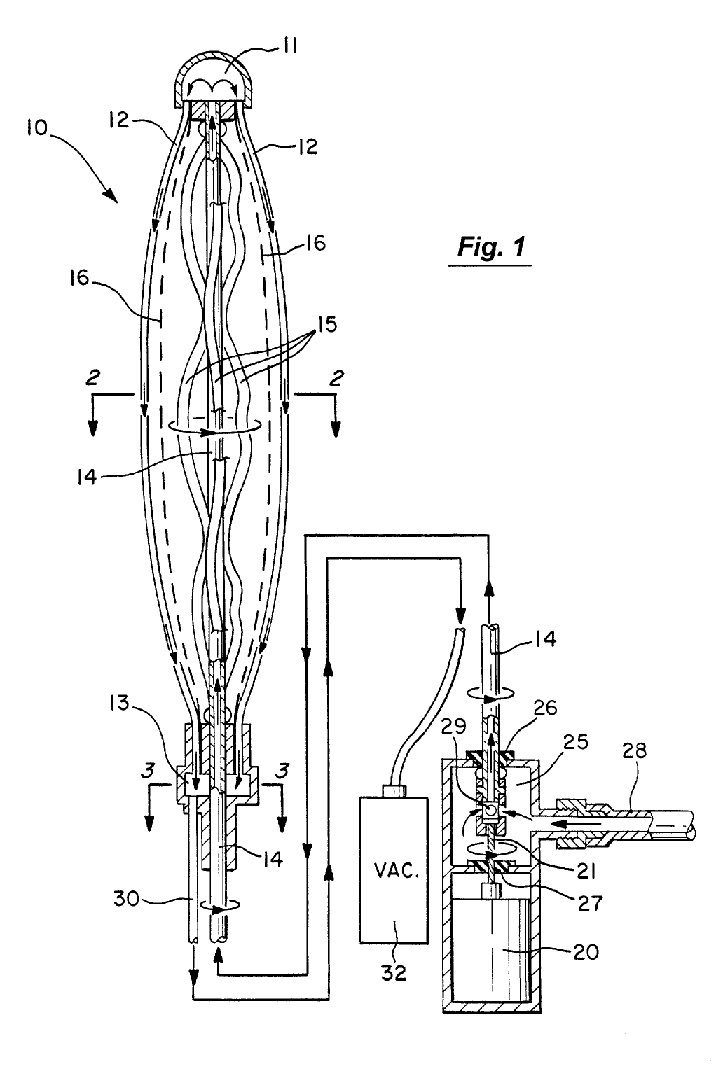

Turning to FIG. 1, a side cross-sectional view is shown an intravenous oxygenator 10 having a plurality of hollow gas-permeable fibers 12. One end of each fiber 12 is potted into a distal manifold 11 and the other end of the fibers is potted into a proximal manifold 13, so that gas can flow between the manifolds 11 and 13 through the fibers 12. However, the ends of each fiber 12 are sealed in fluid communication with the manifolds 11 and 13 so that no gas can escape directly into the surrounding blood stream. In the preferred embodiment, the fibers 12 are formed into a number of relatively flat mats that extend in layers between the manifolds 11 and 13. For example, individual fibers 12 can be bonded together with adhesive or threads to form these mats. The fibers 12 and manifolds 11, 13 define a substantially enclosed region.

The gas-permeable walls of the fibers 12 provide a large total surface area for diffusion of oxygen into the blood stream, and for diffusion of carbon dioxide out of the blood stream. Any of a variety of flexible, hollow, gas-permeable fibers currently available on the market, such as Mitsubishi KPF190M polypropylene fibers, are suitable for this purpose. To provide a true ideal membrane, the polypropylene fibers should be coated with an ultra-thin coating of a gas-permeable polymer (e.g., silicone rubber having a thickness of less than 1 micron) and bonded with a non-thrombogenic component (e.g., heparin).

The distal manifold 11 can be molded from plastic or rubber around the ends of the fibers 12 to prevent the escape of gases at the junction between the fiber ends and the distal manifold 11. For example, as shown in FIG. 1, the distal manifold 11 can be formed as a tapered tip that is contoured to ease insertion of the oxygenator 10 through an incision. The proximal manifold 13 is shown in the cross-sectional view provided in FIG. 3. A vacuum pump 32 can be connected to the vacuum port 30 of the proximal manifold 13, as illustrated in FIG. 1, to enhance the flow of gases through the fibers 12.

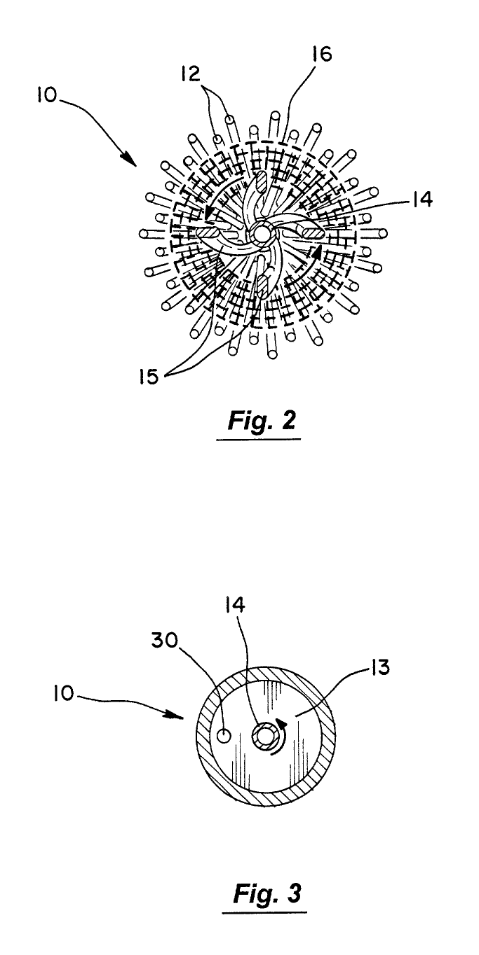

A hollow, rotatable, central shaft 14 extends through the proximal manifold 13 and then passes through the enclosed region and into the interior of the distal manifold 11. The central shaft 14 has at least one hollow lumen extending along its entire length that allows oxygen to be distributed through the central shaft 14 to the distal manifold 11 as illustrated in FIG. 1. A cross-sectional view of the upper portion of the oxygenator 10 is provided in FIG. 2.

An impeller 15 having a plurality of curved blades protrudes radially outward from the central shaft 14 within the enclosed region bounded by the manifolds 11, 13 and fibers 12. The impeller 15 can be made in a variety of configurations, including various corkscrew, paddle, or propeller arrangements. The impeller 15 can also be made of a wide range of materials, including plastics (Teflon, polypropylene, polyurethane, etc), rubber, or various metals. The central shaft 14 extends through the proximal manifold 13 and is connected by a motor 20. The motor 20 rotates the central shaft 14, which in turn spins the impeller 15 to create turbulent blood flow. In the preferred embodiment, the impeller 15 spins at high velocity (e.g., up to 6,000 to 10,000 revolutions per minute). Its configuration is selected to drive blood forward by spinning in a clockwise direction or drive blood in the opposite direction by spinning in a counterclockwise direction. The action of the impeller 15 also drives blood radially through the fiber mat, thereby improving the distribution of blood within the fibers 12 and enhancing the exchange of oxygen and carbon dioxide.

Alternatively, the blades on the impeller 15 can be curved in a substantially spiral fashion with blades on a distal half of the impeller 15 being a mirror image of blades on a proximal half of the impeller 15. In this manner, rotation of the impeller 15 will cause the blades to simultaneously direct blood radially outwardly in two opposite directions. In other words, the blades on the proximal end of the impeller 15 will throw the blood in one radial direction while the blades at the distal end of the impeller 15 will throw the blood in the opposite radial direction.

In the preferred embodiment, the impeller 15 sits within the enclosed region at the center of the hollow fiber mats which are wrapped in concentric circles around the impeller 15. To prevent the impeller 15 from damaging the inner-most layer of the fibers 12, the distal manifold 11 supports a generally cylindrical, porous, protective layer or cage 16 that is slightly larger in diameter than the diameter of the impeller 15 so that the impeller 15 is free to rotate within the protective layer 16. The proximal end of the protective layer 16 is connected to the proximal manifold 13 as shown in FIG. 1. The protective layer 16 separates the fibers 12 from the impeller 15 to prevent the fibers 12 from being entangled in the impeller 15 as it rotates, but allows the free flow of blood through numerous perforations in the protective layer 16. The protective layer 16 is preferably made of a suitable plastic (e.g., Teflon). This protective layer 16 is also very pliable so that the device can be reduced in size for insertion easily without resistance.

To summarize, oxygen-containing gases flow from an external supply through the central shaft 14, into the distal manifold 11, through the fibers 12, and are then exhausted through the exhaust lumen in the proximal manifold 13. Thus, the central shaft 14 serves both to: (1) act as the axis for supporting and spinning the impeller 15; and (2) provide a lumen for delivering oxygen to the distal manifold 11. The central shaft 14 also acts as a structural support for the distal manifold 11 and fibers 12, and provides a degree of rigidity to aid initial insertion of the oxygenator 10 into the blood vessel.

The lower right portion of FIG. 1 depicts the assembly used to simultaneously drive the central shaft 14 and supply oxygen. The proximal end of the central shaft 14 is enclosed in an oxygen supply manifold 25. Oxygen is supplied to the oxygen supply manifold 25 from an external oxygen source through a port 28. An electric motor 20 drives the central shaft 14 by means of a short cable or shaft 21 that passes through a seal 27 into the oxygen supply manifold 25 and is secured to the proximal end of the central shaft 14. A second seal 26 in the opposing wall of the oxygen supply manifold allows the central shaft 14 to freely rotate when driven by the motor 20. A series of openings 29 in the proximal end of the central shaft 14 enable oxygen to flow from the oxygen supply manifold 25 into the lumen of the central shaft 14.

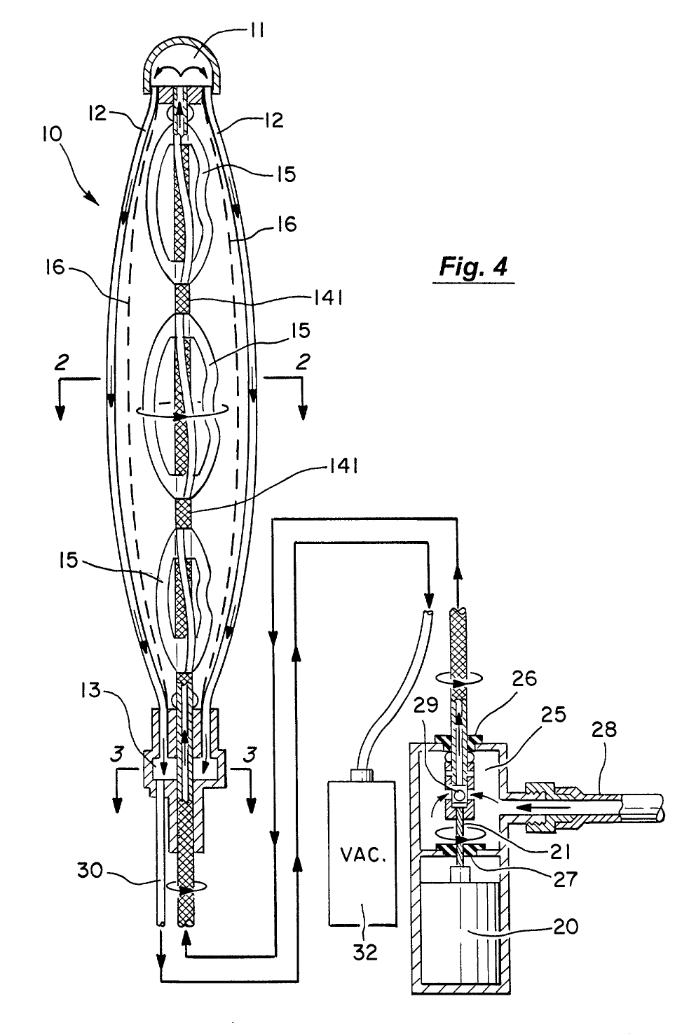

FIG. 4 is a cross-sectional view of another embodiment of the oxygenator 10 in which a hollow, flexible cable 141 is used in place of the central shaft 14 in FIG. 1. The flexible cable 141 has a substantially air-tight central lumen that supplies oxygen to the distal manifold 11 in the same manner as previously described. Thus, either a rigid central shaft 14 or a flexible cable 141 could be employed as a support member to provide structural support for the distal manifold 11, to drive the impellers 15, and to deliver oxygen to the distal manifold 11.

The impeller 15 in the embodiment shown in FIG. 4 is divided into a plurality of discrete segments that are spaced at intervals along the length of the flexible cable 141 within the fiber sheath 12. The flexible cable 141 provides a degree of torsional flexibility and longitudinal flexibility between the impeller segments 15. For example, this flexibility simplifies insertion of the oxygenator 10 into a blood vessel and helps to minimize patient trauma.

In use, the distal portion of the oxygenator 10 is implanted in the venous system of the patient through a single small incision. For example, the device 10 can be implanted through the right internal jugular vein into the superior vena cava and right atrium of a patient. For maximum effectiveness, the distal manifold 11 and fibers 12 are fully inserted through the incision up to the level of the proximal manifold 13. Insertion of the oxygenator 10 can be aided by using a conventional introducer similar to the type presently employed to insert a cardiac pacemaker.

After the device has been implanted, a supply of oxygen-containing gas is connected to the oxygen supply manifold 25 via port 28. The oxygen flows through lumen of the central shaft 14 into the distal manifold 11 and through the fibers 12. Oxygen flows along the interior passageways of the fibers 12 and diffuses outward through the gas-permeable walls of the fibers 12 into the surrounding blood stream. Carbon dioxide also diffuses inward from the blood stream through these gas-permeable walls into the interior of the fibers 12. Carbon dioxide and any remaining oxygen in the fibers are vented to the atmosphere at the proximal ends of the fibers through the proximal manifold 13. Negative pressurization can be applied by means of a suction pump 32 connected to the proximal manifold 13 to enhance gas flow through the fibers 12.

In addition, after the oxygenator 10 has been inserted into the blood vessel up to the location of the proximal manifold 13, the motor 20 is energized to start the central shaft 14 rotating so that the impeller blades 15 will disrupt the linear flow of the blood and move the blood radially in swirling convective flow patterns thereby forcing the fibers 12 surrounding the impeller 15 into continual movement to optimally expose the surface area thereof to the blood thereby maximizing the cross diffusion of gases in and out of the fibers 12. In a preferred use of the device, oxygen is introduced to the fibers 12 at a flow rate of approximately 1 to 3 liters per minute and at a nominal pressure of approximately 6 to 15 mm Hg. A suction pressure of approximately −150 to −250 mmHg is applied at the proximal manifold 13. An alternate configuration would allow the oxygen to be delivered to the proximal manifold and the vacuum to be delivered at the distal manifold.

It should be noted that the present invention can also be used to administer anesthetic gases, gases such as nitric oxide (NO), or other medications directly into the patient's blood system. For this purpose, a mixture of oxygen and anesthetic gases flow through the fibers 12 and diffuse into the patient's blood stream.

Although the present invention has been described with a certain degree of particularity, it is understood that the present disclosure has been made by way of example, and changes in detail or structure may be made without department from the spirit of the invention

I claim:

1. An oxygenator for insertion into a blood vessel comprising:
a distal manifold;
a proximal manifold;
a plurality of hollow gas-permeable fibers extending between the distal manifold and the proximal manifold, said fibers permitting diffusion of gases between blood in the blood vessel and the interior of the fibers;
a rotatable support member extending into the distal manifold and having a lumen in communication with the distal manifold so that oxygen-containing gases flow through the support member, distal manifold, fibers, and proximal manifold; and
an impeller attached to and rotated by the support member to enhance the flow of blood around the fibers.

2. The oxygenator of claim 1 wherein the support member extends through the proximal manifold.

3. The oxygenator of claim 1 further comprising a motor rotating the support member and impeller.

4. The oxygenator of claim 1 further comprising a porous protective layer between the impeller and the fibers.

5. The oxygenator of claim 1 further comprising a suction pump drawing gases through the proximal manifold, fibers, distal manifold and support member.

6. The oxygenator of claim 1 wherein oxygen-containing gases are supplied to the distal manifold through the support member.

7. The oxygenator of claim 1 wherein the support member comprises a hollow shaft.

8. The oxygenator of claim 1 wherein the support member comprises a hollow flexible cable.

9. The oxygenator of claim 1 wherein the fibers form at least one mat substantially enclosing a region between the distal manifold and the proximal manifold containing the impeller.

10. An oxygenator for insertion into a blood vessel comprising:
a distal manifold;
a proximal manifold;
a plurality of hollow gas-permeable fibers extending between the distal manifold and the proximal manifold and defining a substantially enclosed region between the proximal manifold and the distal manifold, said fibers permitting diffusion of gases between blood in the blood vessel and the interior of the fibers;
a rotatable support member extending through the proximal manifold and the enclosed region and into the distal manifold, said support member having a lumen in communication with the distal manifold so that oxygen-containing gases flow through the support member, distal manifold, fibers, and proximal manifold;
an impeller attached to and rotated by the support member within the enclosed region to enhance the flow of blood around the fibers; and
a porous protective layer between the impeller and the fibers.

11. The oxygenator of claim 10 further comprising a motor rotating the support member and impeller.

12. The oxygenator of claim 10 further comprising a suction pump drawing gases through the proximal manifold, fibers, distal manifold and support member.

13. The oxygenator of claim 10 wherein oxygen-containing gases are supplied to the distal manifold through the support member.

14. The oxygenator of claim 10 wherein the support member comprises a hollow shaft.

15. The oxygenator of claim 10 wherein the support member comprises a hollow flexible cable.

16. An oxygenator for insertion into a blood vessel comprising:
a distal manifold;
a proximal manifold;
a plurality of hollow gas-permeable fibers extending between the distal manifold and the proximal manifold and defining a substantially enclosed region between the proximal manifold and the distal manifold, said fibers permitting diffusion of gases between blood in the blood vessel and the interior of the fibers;
a rotatable, flexible cable extending through the proximal manifold and the enclosed region into the distal manifold; said flexible cable having a lumen in communication with the distal manifold so that oxygen-containing gases flow through the flexible cable, distal manifold, fibers, and proximal manifold; and
an impeller rotated by the flexible cable within the enclosed region to enhance the flow of blood around the fibers, said impeller having a plurality of segments attached in series along the flexible cable.

17. The oxygenator of claim 16 wherein the fibers form at least one mat.

18. The oxygenator of claim 16 further comprising a motor rotating the support member and impeller.

19. The oxygenator of claim 16 further comprising a suction pump drawing gases through the proximal manifold, fibers, distal manifold and support member.

20. The oxygenator of claim 16 further comprising a porous protective layer between the impeller and the fibers.
